(19) United States
(12) Patent Application Publication
(10) Pub. No.: US 2016/0128670 A1
(43) Pub. Date: May 12, 2016

(54) LOCKING MECHANISM FOR ULTRASOUND CONDUCTIVE MEDIUM

(71) Applicant: G. Chad MORGAN, Chattanooga, TN (US)

(72) Inventor: G. Chad MORGAN, Chattanooga, TN (US)

(21) Appl. No.: 14/935,901

(22) Filed: Nov. 9, 2015

Related U.S. Application Data
(62) Division of application No. 14/577,397, filed on Dec. 19, 2014.
(60) Provisional application No. 62/077,584, filed on Nov. 10, 2014.

(51) Int. Cl. A61B 8/00

(52) U.S. Cl. CPC A61B 8/4281; A61B 8/4455

(57) ABSTRACT

Embodiments associated with a gel locking mechanism for ultrasound devices are described. The locking mechanism is formed as an attached adaptor or as part of an ultrasound device. In one embodiment, an adaptor device includes a housing formed with a mounting end and a gel receiving end; wherein the gel receiving end includes a side wall formed from the housing that defines a gel cavity for receiving a solidified gel. The adaptor device includes a cap formed with a frame and having a central opening defined therethrough, wherein the frame is configured to lock to and unlock from the gel receiving end of the housing.

### CROSS REFERENCE TO RELATED APPLICATIONS

This patent disclosure claims the benefit of U.S. Provisional Patent Application Ser. No. 62/077,584 entitled “Attachable Adaptor with Locking Mechanism for Ultrasound Conductive Medium,” filed on Nov. 10, 2014, and claims the benefit of U.S. patent application Ser. No. 14/577,397 entitled “Ultrasound Conductive Medium with Locking Element” filed on Dec. 19, 2014, which are all hereby wholly incorporated by reference in their entirety.

### BACKGROUND

In the medical field, ultrasound devices operate with frequencies from 0 to 200 mHz up to several gigahertz. Ultrasound devices may be used for therapeutic procedures by stimulating a target (e.g., tissue beneath the skin's surface) using very high frequency sound waves and/or for generating images of internal structures of a target.

Ultrasound is applied using a device that includes a transducer or applicator that is placed against a patient's skin (via gel). Liquid gel is used on all surfaces of the device's head to reduce friction and to assist transmission of the ultrasonic waves. The liquid gel is squeezed out of a bottle and spread over the patient's skin. Since the gel is in a liquid form, the gel is difficult to contain within a desired area of the skin and the thickness of the gel cannot be controlled. Lack of consistent and desired thickness of the gel leads to an inconsistent coupling distance between the ultrasound applicator and the target surface, which detrimentally affects an ultrasound procedure whether for imaging or therapeutic purposes. When the ultrasound procedure is completed, the patient is required to wipe off and clean the gel from the patient's skin. Typically, the gel is not completely removed and the cleaning process is uncomfortable.

Additionally, liquid gel has been stored in large containers that are typically heated. Heating the gel makes it more comfortable for a patient when the gel is applied. However, the heat and long term storage of such containers creates issues with sterility.

### SUMMARY

In one embodiment, an adaptor device for attachment to an ultrasound device is described. In one embodiment, the adaptor device comprises a housing formed with a mounting end and a gel receiving end; a mounting cavity formed in the mounting end, wherein the mounting end is shaped to mount onto an end of the ultrasound device; wherein the gel receiving end includes a side wall formed from the housing that defines a gel cavity for receiving a solidified gel; a cap formed with a frame and having a central opening defined therethrough, wherein the frame is configured to lock to and unlock from the gel receiving end of the housing; and wherein in a locked position, the cap is closed onto the gel receiving end and the central opening allows the solidified gel inserted into the gel cavity to have a portion extend through the central opening.

In another embodiment, the housing and the cap are connected by at least one hinge, wherein the cap is configured to pivot at the at least one hinge.

In another embodiment, the housing includes a means for connecting the cap with the housing (i.e., one or more hinges, retractable posts, a flap, a cord, or a flip-top).

In another embodiment, the gel receiving end includes an inner ledge formed within the housing, wherein the inner ledge is configured to hold the solidified gel when the solidified gel is inserted into the gel cavity.

In another embodiment, the cap includes one or more latch tabs formed on the frame and wherein the gel receiving end of the housing includes one or more latch recesses positioned to connect with the one or more latch tabs; wherein the frame is configured to lock to and unlock from the gel receiving end of the housing by connecting the one or more latch tabs to the one or more latch recesses.

In another embodiment, an adaptor with a locking mechanism for an ultrasound device is disclosed. The adaptor comprises a housing configured to attach the adaptor to a head of the ultrasound device; a cavity formed by the housing that is configured to receive and contain an ultrasound conductive medium that is in a solidified form; and a hinged locking mechanism for locking the solidified form of the ultrasound conductive medium into the cavity by closing the hinged locking mechanism over the cavity and against the housing.

In another embodiment, the hinged locking mechanism is configured to unlock the solidified form of the ultrasound conductive medium from the cavity by disconnecting the hinged locking mechanism from the housing.

In another embodiment, the hinged locking mechanism includes a cap formed with a frame and having a central opening formed through the frame, wherein the frame is connected to the housing by at least one hinge.

In another embodiment, the hinged locking mechanism includes one or more latch tabs and wherein the housing of the adaptor includes one or more latch recesses corresponding to the one or more latch tabs; wherein the hinged locking mechanism is configured to lock to and unlock from a side wall of the housing by connecting the one or more latch tabs to the one or more latch recesses.

In another embodiment, the cavity includes an inner ledge formed by the housing, wherein the inner ledge extends around a perimeter of the cavity and is configured to hold the solidified form of the ultrasound conductive medium when the solidified form is inserted into the cavity.

In another embodiment, the adaptor is a retrofit component for attachment to the ultrasound device.

In another embodiment, an ultrasound device with a gel locking mechanism is disclosed. The ultrasound device comprises a housing including a handle and a head extending from the handle; an applicator surface formed on the head; at least one transducer for generating ultrasound energy, wherein the at least one transducer is mounted within the housing and adjacent the applicator surface; a side wall formed from the housing and extending from the head and around a perimeter of the applicator surface to define a gel cavity; and a cap formed with a frame and having a central opening defined therethrough, wherein the frame is configured to lock to and unlock from the side wall; and wherein in a locked position, the cap is closed onto the side wall to secure a piece of solidified gel that is inserted into the gel cavity and wherein the central opening allows the solidified gel inserted into the gel cavity to have a portion extend through the central opening.

In one embodiment, the head and the cap are connected by at least one hinge.

In another embodiment, the solidified gel is inserted into the gel cavity; and wherein in the locked position, the frame of the cap is connected to the side wall and encloses at least sides of the gel cavity and is in contact with outer edges of the solidified gel.

In another embodiment, the cap includes one or more latch tabs formed on the frame and wherein the side wall of the housing includes one or more latch recesses corresponding to the one or more latch tabs; wherein the frame is configured to lock to and unlock from side wall of the housing by connecting the one or more latch tabs to the one or more latch recesses.

### BRIEF DESCRIPTION OF THE DRAWINGS

The accompanying drawings, which are incorporated in and constitute a part of the specification, illustrate various systems, methods, and other embodiments of the disclosure. It will be appreciated that the illustrated element boundaries (e.g., boxes, or other shapes) in the figures represent one embodiment of the boundaries. In some embodiments one element may be designed as multiple elements or that multiple elements may be designed as one element. In some embodiments, an element shown as an internal component of another element may be implemented as an external component and vice versa. Furthermore, elements may not be drawn to scale.

FIG. 1A illustrates one embodiment of an ultrasound device shown with components unassembled including in embodiment of an adaptor.

FIG. 1B illustrates the adaptor of FIG. 1A attached to the ultrasound device in an assembled view.

FIG. 2A illustrates a top view of one embodiment of the adaptor.

FIG. 2B illustrates a side view of one embodiment of the adaptor.

FIG. 2C illustrates a perspective view of one embodiment of the adaptor.

FIGS. 3A, 3B, and 3C illustrate top, perspective, and side views of another embodiment of a preconfigured solidified gel configured with a locking ring.

FIGS. 4A, 4B, 4C, and 4D illustrate a top view, left side view, right side view, and bottom view, respectively, of another embodiment of an adaptor including a locking recess.

FIG. 4E illustrates a cross-section view of the adaptor through section A-A of FIG. 4B.

FIGS. 5A-5E illustrate multiple views of another embodiment of an attachable adaptor including a locking mechanism.

FIG. 5F illustrates a side view of the attachable adaptor of FIGS. 5D and 5E with the solidified gel locked in and extending through an opening of the cap.

FIG. 5G illustrates a side view in cross-section view of the attachable adaptor of FIG. 5E without the solidified gel positioned therein.

FIG. 6 illustrates one embodiment of an assembly view of the adaptor from FIG. 5A being attached to the head/nose of an ultrasound device/probe (e.g., the device 100 from FIG. 1A).

FIG. 7 illustrates a side view of another embodiment of the attachable adaptor of FIGS. 5A-5G with a removable cap/top portion for locking the solidified gel to the base portion.

FIG. 8 illustrates one embodiment of an ultrasound device formed with a gel locking mechanism.

### DETAILED DESCRIPTION

Various embodiments of a locking mechanism for locking a solidified piece of ultrasound conductive medium to an ultrasound device is disclosed. In one embodiment, the locking mechanism is configured as an adaptor that is attachable to an ultrasound device (e.g., an imaging or therapeutic ultrasound device). In another embodiment, the locking mechanism is formed as part of an ultrasound device. The locking mechanism functions to allow for loading, unloading, and replacement of solidified pieces of ultrasound conductive medium for the ultrasound device.

Using an ultrasound conductive medium that is solidified provides for a consistent coupling medium for an ultrasound procedure, which improves the application of ultrasound to a target. The present locking mechanism improves the ability to handle and control the solidified medium, and to prevent the solidified medium from falling out of the ultrasound device during a procedure.

In one embodiment, the attachable adaptor is configured to attach to an applicator tip/head of an ultrasound device/probe. Once attached, the adaptor converts a flat surface of the head (e.g., the applicator surface that is placed against the skin of a patient) to a surface that includes a gel cavity/receptacle. The gel cavity is configured to receive and maintain a portion of a solidified conductive medium (i.e., ultrasound gel in a solidified state) that is formed/preconfigured with a shape to fit into the gel cavity. Thus the adaptor converts the physical form of an ultrasound device/probe to a different physical form that can self-contain a solidified piece of ultrasound gel and lock the solidified gel in place, and unlock to release the solidified piece for replacement. In one embodiment, the adaptor is a retrofit component for attachment to an existing ultrasound device/applicator.

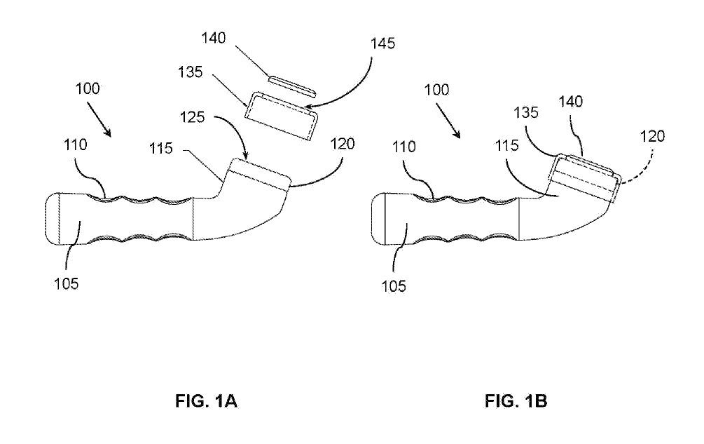

With reference to FIGS. 1A and 1B, one embodiment of an ultrasound device 100 is shown in a partially unassembled state (FIG. 1A) and in an assembled state (FIG. 1B). The device 100 is configured as a hand-held device formed with a housing that includes a handle 105 with zero or more finger grips 110 (e.g., indentations, ridges, and so on). The handle 105 (e.g. handle end) is connected to a head portion 115 (e.g. applicator end of the housing) that includes one or more sides that connect to or forms a nose 120, where inside the housing within the area of the nose 120 contains one or more ultrasound transducers therein (not shown). The head 115 and nose 120 are sometimes referred to as simply the “head,” “nose,” or “transducer.”

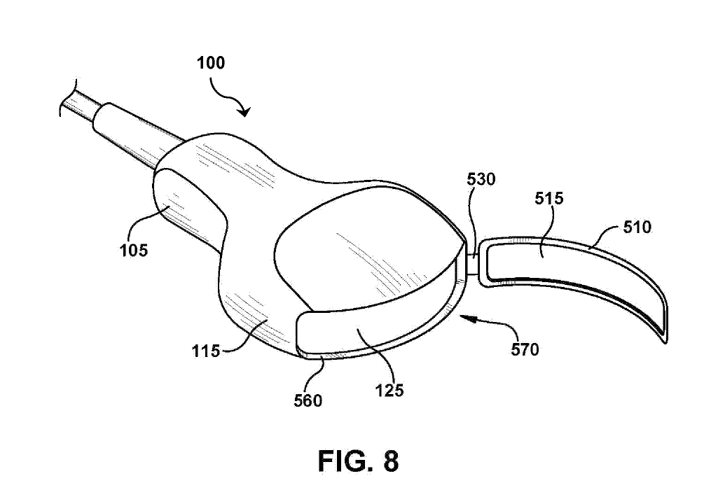

In the illustrated embodiment, the nose 120 includes a generally flat applicator surface 125, which is the surface that is put in contact with a target (e.g., ultrasound patient, diagnostic object) via gel. The applicator surface 125 may have a variety of shapes depending on the designed shape of the head 115. The shape may be circular, oval, elliptical, rectangular, variations of these shapes, or other implemented shape. In some embodiments, the applicator surface 125 may be straight or curved (e.g., convex shape) from one side to the other (see for example FIG. 8, curved applicator surface 125).

In one embodiment, nose 120 is configured with a connector (not shown) that is threaded to insert and connect with a corresponding threaded socket in the head 115. In another embodiment, the connector may be configured as a quick-connect/disconnect device so that the nose 120 can be connected to the head 115 by pushing and snapping into place or disconnected by pulling off with a small amount of force.

Thus, in one embodiment, the nose 120 is configured as a removable and replaceable component. In another embodiment, the head 115 and nose 120 are integral with each other and formed with the same housing. In general, the nose 120 is the portion that contains a transducer (e.g., piezoelectric crystal disposed within the nose) that generates ultrasound energy, which is transmitted through the applicator surface 125. Of course, any form of ultrasound device including a device with multiple transducers can be used.

With further reference to FIG. 1A, in one embodiment, an attachable adaptor 135 is shown that is configured to convert the flat surface 125 of the ultrasound device 100 to have a receptacle/cavity 145 for receiving and holding a preconfigured ultrasound conductive medium 140 (i.e., a piece of solidified gel shaped to fit into the cavity 145). In other words, the receptacle/cavity 145 is a defined containment area for the solidified gel 140.

For example, the adaptor 135 is configured to attach to the head portion 115/120 of the device 100 and convert the head portion 115/120 from an existing configuration (e.g., first state having a flat surface) that does not have a gel receiving cavity to a different configuration (e.g., second state that is a non-flat surface) that includes a gel receiving cavity. In one embodiment, the housing of the adaptor 135 is configured to attach to the nose 120 and be positioned between the nose and the ultrasound conductive medium 140. In one embodiment, the components are in a stacked relationship, for example, (1) the ultrasound device, (2) the adaptor, and (3) the solidified ultrasound conductive medium, which overlaps with the adaptor.

After the adaptor 135 is attached, the ultrasound device 100 no longer has a flat applicator surface 125 that uses liquid gel during an ultrasound procedure. Rather, the solidified conductive medium 140 is inserted into the adaptor 135, which holds the solidified conductive medium 140 in place. The solidified conductive medium 140 provides for a predetermined, consistent, and controlled amount/thickness of ultrasound conductive medium as compared to the random, uncontrolled amounts of liquid gel that are used.

In one embodiment, the adaptor 135 is configured as a cap structure so that it can be clipped-on, snapped-on, slid-on, attached to, or otherwise connected over the head/nose 120 by pushing on and/or snapping into place. The adaptor 135 may also be disconnected by pulling off with a small amount of force. Thus in one embodiment, the adaptor 135 is an attachable and detachable component.

The shape and size of the adaptor 135 is configured to correspond to the configuration of the head/nose 120 of a targeted ultrasound device for which the adaptor 135 is designed to mate with. Accordingly, a custom fitted adaptor 135 can be formed for different types and models of ultrasound transducers or probes.

FIG. 1B shows the nose 120 inserted into the adaptor 135, or in other words, the adaptor 135 attached to the nose 120. The adaptor 135 may be configured to be held in place by at least surface tension or pressure with surfaces of the nose 120, by friction with the inside surfaces of the adaptor 135, by an adhesive, by mating with corresponding lips or edges from the adaptor 135, and/or from the head 115/120, and/or by a connecting device.

The adaptor 135 is configured with edges or side walls that define a cavity 145 that is configured to receive and contain an ultrasound conductive medium 140 used during an ultrasound or imaging scan. In one embodiment, the preconfigured ultrasound conductive medium 140 is a portion of ultrasound gel that was initially in a flowable state and has been solidified into a solid, non-flowable state. The preconfigured gel 140 is solidified and formed/molded with a shape to correspond to and/or mate with the shape of the gel cavity 145. As seen in FIG. 1B, the gel 140 is inserted into the cavity 145 of the adaptor 135.

The solidified gel 140 and adaptor 135 are configured in a cooperative relationship such that the gel 140 extends a distance beyond the edges of the adaptor 135 once the gel 140 is inserted. For example, the gel 140 includes a thickness that is greater than the sidewalls of the cavity 145. Thus, the gel 140 is positioned to provide an interface and coupling medium between the applicator surface 125 of the device 100 and a patient's skin where the gel 140 can contact the skin.

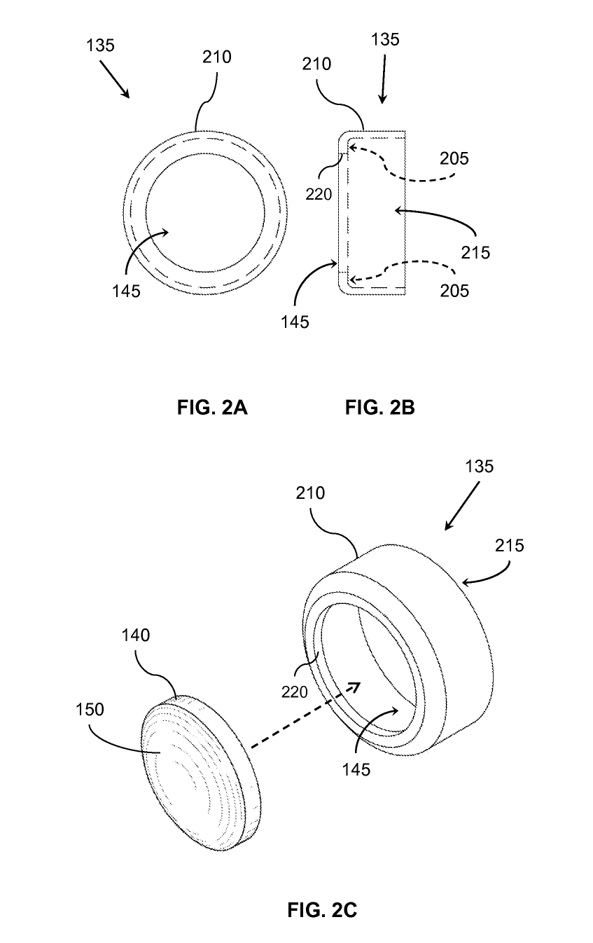

In one embodiment, the adaptor 135 holds the preconfigured gel 140 so that the preconfigured gel 140 is disposed directly on the applicator surface 125 of the nose 120. The preconfigured gel 140 is formed to generally correspond to the shape of the receptacle/cavity 145 of the adaptor 135 so the gel 140 fits into the cavity 145. Thus in one embodiment, the adaptor 135 includes two cavities: one for receiving the ultrasound device nose 120 (shown as cavity 215 in FIGS. 2B and 2C) and one for receiving the preconfigured gel 140 (cavity 145). The adaptor 135 is explained in more detail with reference to FIGS. 2A-2C.

With reference to FIGS. 2A-C, one embodiment of the adaptor 135 is shown in a top view in FIG. 2A, a side view in FIG. 2B, and a perspective view in FIG. 2C along with the preconfigured gel 140.

In one embodiment, the adaptor 135 includes a housing formed in a generally cap-like structure that can fit over the head 115 of the ultrasound device 100 and over the applicator surface 125. In another embodiment, the adaptor 135 has generally a tube structure.

The adaptor housing is made from a material that can operate with ultrasound energy generated from the transducer of the ultrasound device 100. The material may be metal, metallic, polymer, plastic, or other material that functions with ultrasound energy so as to have minimal disruptive or interference effects from the intended operation of the ultrasound device. In another embodiment, the adaptor 135 is made using material that can act as an insulator of ultrasound energy so as to function as a directional control component to direct ultrasound energy from the ultrasound device to be transmitted out from the cavity 145 of the adaptor 135.

With reference to FIG. 2A, the adaptor 135 is configured with a circular shape defined by an exterior sidewall 210 that forms the housing. The adaptor 135 is circular since the targeted ultrasound device 100 has a circular head 115. Of course the shape of the adaptor 135 will be different for a differently shaped ultrasound device.

When connected to the ultrasound device 100, a cavity sidewall 220 at one end of the sidewall 210 is configured to extend out from the applicator surface 125 (see FIG. 2B, FIGS. 1A-1B) and define the gel receiving cavity 145. The gel cavity 145 has a perimeter defined by the cavity sidewall 220 on a first end of the adaptor 135 (e.g., gel side).

In one embodiment, the housing of the adaptor 135 includes an inside surface 205 that defines an edge or stop. When the adaptor 135 is attached to the ultrasound device 100, the nose 120 of the ultrasound device is inserted through an opposite second end of the adaptor that defines an opening 215. The nose 120 would slide into the adaptor 135 and contact against the surface 205, which would stop the nose 120 from sliding through the entire opening of the adaptor 135. Of course, other configurations of a stop mechanism can be implemented (e.g., internal ribs or edges, etc.). In another embodiment, the housing is configured as a tube housing that tapers toward one end that functions as the stop mechanism. Thus the narrowing of the tube causes contact with the ultrasound head thereby locking the adaptor 135 in place (e.g., tight fit). Accordingly, the inside surface of the housing of the adaptor 135 does not have an inner stop edge.

Once inserted into the adaptor 135, the exterior applicator surface 125 of the nose 120 (see FIG. 1B) is exposed in the gel cavity 145 and forms the bottom surface of the cavity 145. Thus, when the preconfigured piece of gel 140 is inserted into the cavity 145, the preconfigured gel 140 lays against the applicator surface 125 of the nose 120. The cavity sidewall 220 functions to hold the preconfigured gel 140 from moving or falling off the ultrasound device.

In one embodiment, the height of the cavity sidewall 220 is less than the thickness of the preconfigured gel 140. In this manner, the top exposed surface 150 of the gel 140 extends beyond the housing of the adaptor 135 when the gel 140 is inserted in the cavity 145 in order to contact an object of interest (e.g., skin) (see FIG. 2C and FIG. 1B).

In another embodiment, the inside surface 205 may be a dividing wall that extends across the adaptor 135. Thus the entire wall acts as a stop mechanism. In this manner, the dividing wall 205 would separate the preconfigured gel 140 in the cavity 145 and the applicator surface 125 of the nose 120 that is inserted into the adaptor 135. Thus the applicator surface 125 would not be exposed and would not directly contact the solidified gel 140.

The opening 215 (e.g., the second cavity in the adaptor 135) is configured to fit on (attach to) and join with or connect to the ultrasound device. As previously stated, the adaptor 135 is shown in a generally circular shape for purposes of example only, but other shapes may be implemented based on the shape of the ultrasound device for which the adaptor 135 is configured to fit on (e.g., rectangular, oval, other polygon shape, irregular shape, and so on). In general, once attached to an ultrasound device, the adaptor 135 converts the flat applicator surface 125 of the ultrasound device to a surface that has side walls 220 extending out from the applicator surface 125. The side walls 220 are configured to can contain a preconfigured ultrasound gel and hold the gel in place during an ultrasound procedure (e.g., during therapy, imaging, etc.). In this manner, liquid gel is not needed to be spread over the skin of a patient.

In one embodiment, the sidewalls 210 and 220 are a continuous edge or rim around the perimeter of the cavity 145. In another embodiment, the sidewall 210 and 220 may include one or more notches (not shown). A notch may be used to remove the preconfigured gel 140 from within the cavity 145 by inserting a finger in the notch to access the gel 140 within the cavity 145. In another embodiment, the cavity sidewall 220 may be perforated or be configured as two or more portions such as prongs that can hold a piece of solidified gel.

With reference again to FIG. 1A, in one embodiment, in the inside of the nose 120, the nose 120 includes one or more transducers (e.g., piezoelectric crystal) (not shown) for generating ultrasound waves. The transducer is connected within the nose 120 and secured against an inside surface of the nose 120.

The various dimensions shown are only exemplary of one embodiment. It is not intended to limit the construction of the adaptor 135 shown since the adaptor 135 can be formed with different shapes and sizes.

Ultrasound Device Components

With reference again to FIG. 1A, in one embodiment, the handle 105 and head 115 are formed from a housing that contains one more components (not shown) configured to generate ultrasound energy (e.g., for therapy applications) or to generate and receive/detect ultrasound energy (e.g., for imaging applications). In one embodiment, the ultrasound device 100 includes an energy generating module operative to generate a driving signal that can be transformed into ultrasonic energy. The energy generating module includes a local power source or receives power from a remote source via a power cord, an oscillator, and a driver component. The portable ultrasound device 100 also includes an ultrasound transducer having a piezoelectric component, which is disposed within the nose 120 and is generally near or adjacent to the applicator surface 125. The ultrasound transducer is operative to receive the driving signal from the energy generating module and transform the driving signal into ultrasonic energy. There are many different types of internal components that can be used to implement the ultrasound device 100. Since they are not the focus of the present disclosure, they are not described in detail.

In another embodiment, the device 100 may include an internal memory for storing ultrasound data collected by the device 100. The device 100 may include an interface for communicating the data from the memory to a remote device. The device 100 can be configured to communicate the data via a wire connection and/or a wireless connection.

Preconfigured Ultrasound Conductive Medium

With reference to FIG. 2C, in one embodiment, the preconfigured gel 140 is an ultrasound conductive medium for use with therapeutic or imaging ultrasounds and electrotherapy devices. In one embodiment, the preconfigured gel 140 is formed or molded into a specified solidified shape that fits into and is contained within the cavity 145 of the adaptor 135 for which the gel is made. For example, the preconfigured gel 140 is a solidified form of a jelly-like ultrasound conductive medium. The gel is preconfigured since the ultrasound conductive medium is initially in a flowable state and then has been solidified into a non-flowable state. The solidified form is made to mate with the receptacle/cavity 145 of the retrofitting adaptor 135, which is configured to attach to the head of an ultrasound device.

In one embodiment, a process to form the preconfigured gel 140 may involve using molds. Multiple molds can be used to create multiple pieces of the preconfigured gel at a time. For example, a tray of patterned shapes can be used where a gel composition initially in a flowable state (e.g., liquid, jelly-like, or aqueous form) is poured into each patterned shape. The flowable gel composition is then processed to solidify the gel to a desired extent so that its shape is set (e.g., the solidified composition holds its shape, maintains its dimensions, does not flow, and/or does not take the shape of its container when placed in the container (e.g., liquid properties)).

The solidifying process may involve curing, heating, cooling, or other process to solidify the aqueous composition. In different embodiments, the preconfigured gel 140 can be solidified to different degrees or ranges as desired such as being a soft and flexible object, being a rigid object, or any state in between (e.g., semi-rigid, elastic, and/or flexible structure). Of course, other processes may be used to create the preconfigured gel such as injection molding, 3-D printing, and so on.

In one embodiment, the preconfigured gel 140 is maintained within the gel cavity 145 of the adaptor 135. Thus during a scan, the gel 140 moves with the ultrasonic device 100 by being a part of the adaptor 135. The top exposed surface 150 of the preconfigured gel 140 is in contact with a patient's skin and acts as a lubricant to help the ultrasound device 100 slide across the skin from area to area.

Air and other gases may impede sound waves. Thus, the solidified gel 140 functions to prevent the formation of air bubbles between the transducer and the patient's skin and helps conduct sound waves from the transducer into the patient's body. Spreading unknown amounts of liquid gel on a patient is reduced or may be eliminated.

The solidified piece of gel 140 maintains a predetermined thickness of ultrasound conductive medium. The solidified gel 140 thus provides a consistent and controlled thickness of the ultrasound conductive medium between a transducer of the ultrasound device and a patient. The consistent and controlled thickness of gel may improve the quality and/or consistency of the ultrasound energy applied to a patient because the thickness of the solidified gel 140 does not change. Thus the transducer and the object of interest (e.g., skin) are maintained at a consistent distance from each other.

After being used in an ultrasound procedure, the solidified gel 140 may be removed from the adaptor 135 and replaced with a new piece of solidified gel. Of course, the same piece of gel may be used for multiple procedures since it is solidified and moves with the adaptor. However, there may be issues with sterility from multiple uses that may not be desired.

Solidified Gel with Locking Element Embodiment

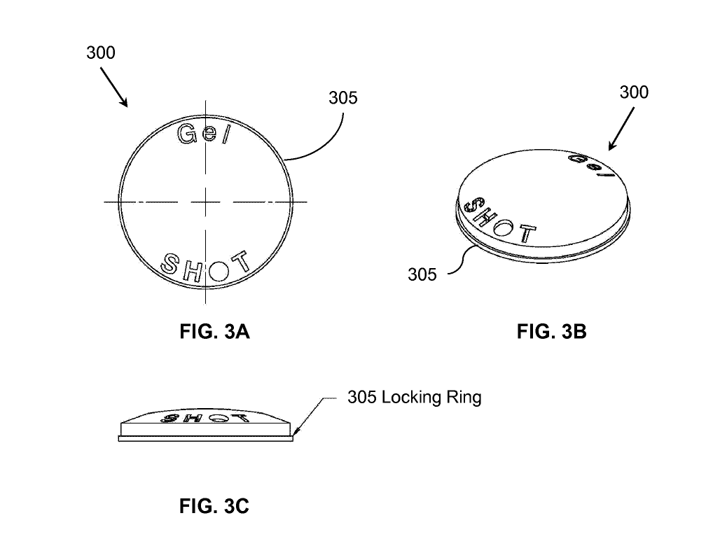

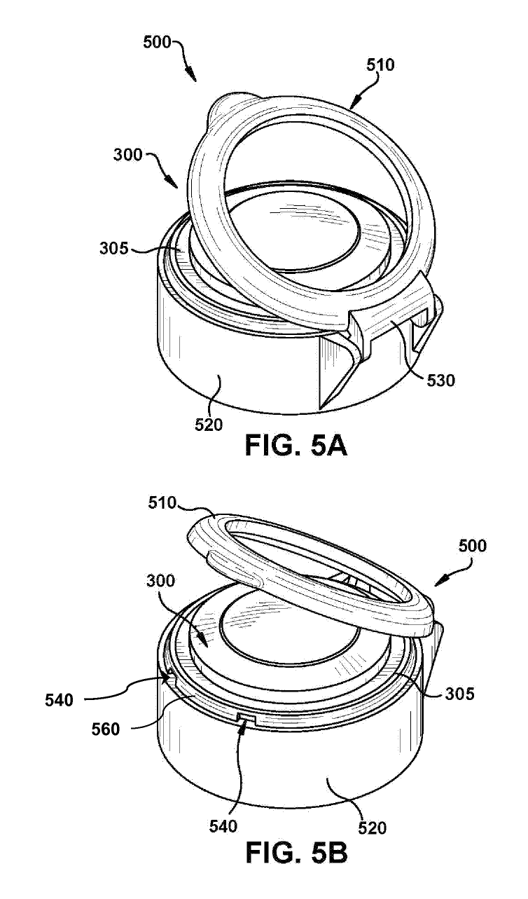

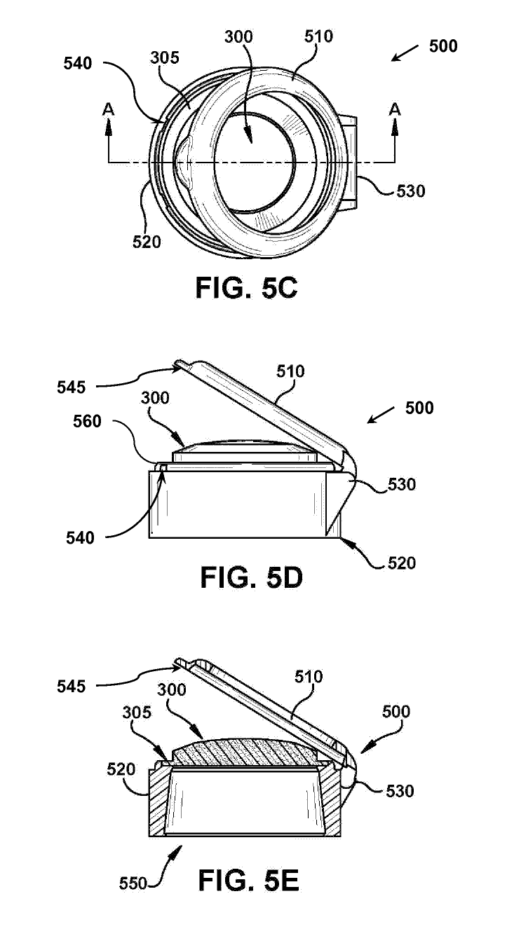

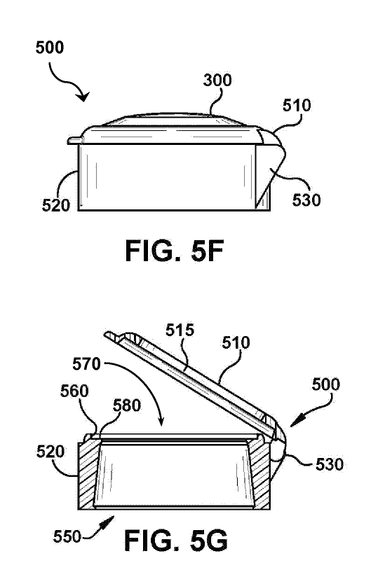

FIGS. 3A, 3B, and 3C illustrate top, perspective, and side views, respectively of another embodiment of a solidified gel 300 configured with a locking element 305. The locking element 305 is generally a protruding portion of the solidified gel 300 that extends out from the gel surface or side wall to help restrict movement of the gel 300 when inserted into a gel cavity of an adaptor (e.g., like adaptor 135). In one embodiment, the solidified gel 300 and locking element 305 will be described as functioning with a gel locking mechanism of FIGS. 5-8.

With reference to FIGS. 3A-3C, in one embodiment, the side wall of the gel 300 is formed with a ring 305 that extends out from the sidewall and extends around the perimeter of the gel 300. From a different perspective, the ring 305 may be formed by having the base of the gel have a larger diameter that the top portion of the gel 300 as seen in the top view of FIG. 3A. The ring 300 may be positioned along any desired location along the height of the sidewall.

In other embodiments, rather than being one continuous ring, the locking ring 305 may be configured as one or more partial rings where each partial ring is less than the circumference of the gel 300. In another embodiment, the locking ring 305 may be configured as one or more ribs, lips, edges, or other protrusions that extend out from the sidewall in horizontal and/or vertical directions and may have any desired shape.

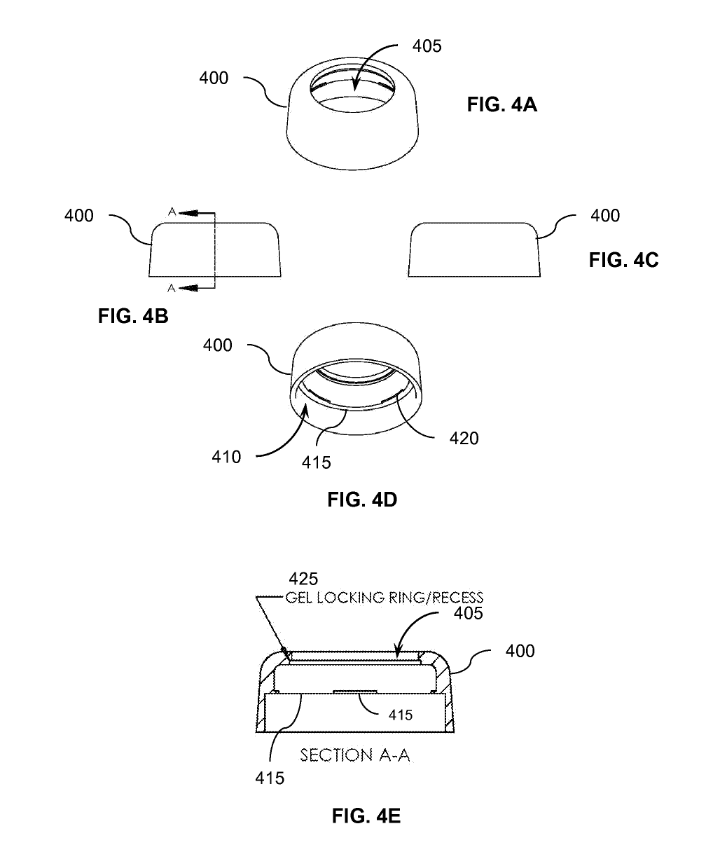

The ring 305 is configured to fit into a corresponding locking ring/recess in a gel adaptor for attachment to an ultrasound device. When the gel 300 is inserted into a gel cavity in the adaptor (e.g., in the adaptor 135 in FIG. 2C, in adaptor 400 in FIG. 4A, in adaptor 500 in FIGS. 5A-5G, or similar cavity), the gel 300 is restricted from unintentionally falling out of the gel cavity.

In another embodiment, the gel 300 is formed or molded in a predefined solidified shape and includes indentations in one or more surfaces. For example, the indentations may be a word or phrase. As seen in FIGS. 3A and 3B, the gel 300 includes the words “Gel Shot” indented on the top surface.

With reference to FIGS. 4A-4E, one embodiment of a gel adaptor 400 is shown in a variety of views. For example, FIG. 4A: top perspective view; FIG. 4B: left side view; FIG. 4C: right side view; FIG. 4D: bottom perspective view; and FIG. 4E: cross-section view of section A-A from FIG. 4B.

The gel adaptor 400 is configured to attach to the head of an ultrasound device (as previously described) that does not have a gel cavity for holding a piece of solidified gel preconfigured with a corresponding shape. Once attached, the adaptor 400 converts the existing flat applicator surface 125 of the ultrasound device 100 to a device with a gel cavity that can receive and hold a piece of solidified gel (e.g., gel 140—FIGS. 1A-1B; gel 300—FIGS. 3A-3C).

The adaptor 400 is configured with a top opening (gel cavity) 405 (FIG. 4A) and a bottom opening 410 (FIG. 4D). The housing of the adaptor 400 is configured with a shape that corresponds to the shape of an ultrasound head so that the bottom opening 410 and sidewalls connect/attached to the ultrasound head. In that regard, the adaptor 400 may be configured with various internal shapes and edges 415 to contact surfaces of the ultrasound head for a better fit. In other embodiments, the internal portion of the adaptor 400 may include one or more lips 420 to provide additional connection points. The solidified gel being used (e.g., preconfigured gel 300 (FIG. 3A-3C)) is formed with a shape to generally match the shape of the top opening/cavity 405. Other shapes may include oval, rectangular, or other polygonal shape.

With reference to FIG. 4E, the adaptor 400 is configured with a gel locking ring/recess 425 within the gel cavity 405. The locking recess 425 is formed to correspond to the locking ring 305 of the solidified gel 300 and is configured to receive/connect/mate with the locking ring 305. Since the solidified gel 300 is a flexible/malleable substance, the gel 300 can be pressed into the cavity 405 until the two shapes align and connect (e.g., the protruding portion 305 of the gel is inserted into the recess 425). Thus, the locking ring 305 inserts into the corresponding locking recess 415 to lock the gel 300 in the cavity 405. Accordingly, the solidified gel 300 is held in the cavity 405.

Overall, in one or more embodiments, the adaptor disclosed herein is a component for retrofitting an existing ultrasound device/applicator (e.g., an operational device that functions without the adaptor). The adaptor modifies an applicator surface of the ultrasound device to have a gel receiving cavity or receptacle. Thus existing ultrasound applicators that are functional can be modified rather than replacing the ultrasound applicator or system with a new system, which is more costly. Furthermore, the use of liquid gel can be eliminated as described herein.

Locking Mechanism for Ultrasound Conductive Medium

With reference to FIGS. 5A-5G, one embodiment of an adaptor device 500 is illustrated in various views. The adaptor device 500 is configured with a gel locking mechanism for receiving and locking a solidified gel to an ultrasound device, and unlocking the solidified gel to allow the solidified gel to be removed and replaced.

As will be described in one embodiment, the gel locking mechanism includes at least a cap 510 that closes onto a housing base 520 to lock a solidified gel 300 and opens to unlock the solidified gel (allowing the gel to be removed and replaced). In one embodiment, the cap 510 is configured as a flip-top that can be opened and closed by pulling, pushing, or flicking with fingers.

FIG. 5A shows a perspective view of the adaptor 500 with a piece of solidified gel 300 loaded within a gel cavity of the adaptor 500. The adaptor 500 is a hinged locking mechanism that locks and unlocks the solidified gel 300 from the adaptor 500. The solidified gel 300 is also shown in FIGS. 3A-3C and includes a locking ring 305 (e.g., protrusion, edge, or lip) that extends from the gel 300. As seen in FIGS. 3A-3C, the gel locking ring 305 extends out from a bottom/side surface of the solidified gel 300 providing an area around the perimeter of the gel 300 for engaging with the cap 510. For example, the cap 510 clamps down on the gel locking ring 305 when closed.

In one embodiment, the adaptor 500 is a device formed with a housing that includes an adaptor top portion 510 (cap) and an adaptor base 520. The base 520 includes a mounting end and a gel receiving end (that holds the gel) on the opposite side. The base 520 is configured to be mounted to an ultrasound device/probe and the cap 510 is configured to lock in the solidified gel 300 onto the base 520 (as will be described below). Once mounted to the head/nose of an ultrasound device/probe, the adaptor converts the physical form of the ultrasound device/probe (i.e., flat surface without a gel cavity) to a different physical form that can contain a solidified piece of ultrasound gel and lock the solidified gel in place.

FIG. 5B shows a perspective view that is rotated from FIG. 5A. Both figures show the adaptor top 510 in an open/unlocked position. FIG. 5C is a top view showing the top portion 510 open with the solidified gel 300 positioned on the base 520 on the gel receiving end. FIG. 5D is a side view showing the top portion 510 open/unlocked and the gel 300 loaded inside the adaptor 500. FIG. 5E is a cross-section view through line A-A (shown in FIG. 5C).

In one embodiment, the top portion 510 is formed as a frame that defines a central opening 515 therethrough (see for example, FIG. 5A shown as a window frame structure). The top portion 510 is a cap or lid that locks and unlocks against the base 520. The cap 510 includes the central opening 515 through which the top surface of the solidified gel 300 extends through once the cap 510 is closed and locked against the base 520 (see for example, FIG. 5F).

In one embodiment, the adaptor 500 includes a hinge 530 that connects the cap 510 to the base 520 and allows the cap 510 to move and pivot relative to the base 520 at the hinge 530. With the hinge 530, the cap 510 is a flip-top. For example, the cap 510 can be opened and thus unconnected and unlocked to the top surface (gel receiving surface/end) of the base 520, or be closed and thus attached/locked to the top surface of the base 520. The cap 510 is similar to a door or lid that opens and closes by pivoting along the hinge 530. In another embodiment, the adaptor 500 includes multiple hinges 530 that connect the cap 510 to the base 520. In another embodiment, the hinge 530 is formed as flap that connects the cap 510 to the base 520 where the flap allows the cap 510 to move and pivot relative to the base 520.

With continued reference to FIG. 5B, in one embodiment, the top portion (cap) 510 includes one or more latch tabs 545 (see FIGS. 5D and 5E) formed on the frame of the cap 510. The latch tabs press fit, snap, or hook into corresponding latch recesses 540 formed on the base 520 (or formed on the side wall 560 that defines the gel cavity, see FIGS. 5B and 5D). The latch tabs lock the cap 510 against the base 520 when the cap 510 is closed and the latch tabs are mated and/or engaged with their corresponding latch recesses. Of course, the latch recesses may be configured as latch tabs or hooks that connect with the latch tabs 545 of the cap 510.

FIG. 5E shows (in cross-section) the top portion cap 510 configured with outer edges of the frame that when closed against the base 520, overlap and press down against the gel lock ring 305 to lock the solidified gel 300 into the adaptor 500. In other words, the cap 510 overlaps the outer edges of the solidified gel 300 to clamp the gel 300 to the base 520. The top portion cap 510 includes a central opening 515 to allow the top surface of the gel 300 to be exposed. The thickness or height of the frame of the cap 510 is less than the thickness or height of the central portion of the solidified gel 300. Since the gel 300 is thicker, the central portion of the gel 300 extends through the opening 515 of the cap 510 (see FIG. 5F). This allows the solidified gel 300 to be pressed into contact with an object (e.g., skin) for an ultrasound procedure.

When using the adaptor 500 with an ultrasound device/application, the gel 300 is exposed to contact the skin of a patient. By opening the cap 510, the solidified gel 300 can be removed from the adaptor 500 and replaced with a new piece of solidified gel by inserting the new solidified gel into the gel receiving cavity of the adaptor 500. By closing the cap 510 against the base 520, the solidified gel 300 is locked against the applicator surface of the ultrasound device since the edges of the cap 510 overlap and engage against the lock ring 305 of the solidified gel 300.

With continued reference to FIG. 5E, the base 520 of the adaptor device is formed with a mounting cavity 550 at the mounting end. The mounting cavity 550 is formed by the housing wall to correspond to the shape of an ultrasound head or applicator tip onto which the adaptor 500 will be attached (e.g., pushed onto, locked, friction hold, snapped onto, etc). Thus, the adaptor 500 can be retrofitted onto an existing device head and convert the head (which has a flat applicator surface) to a surface that carries and uses replaceable pieces of solidified gel.

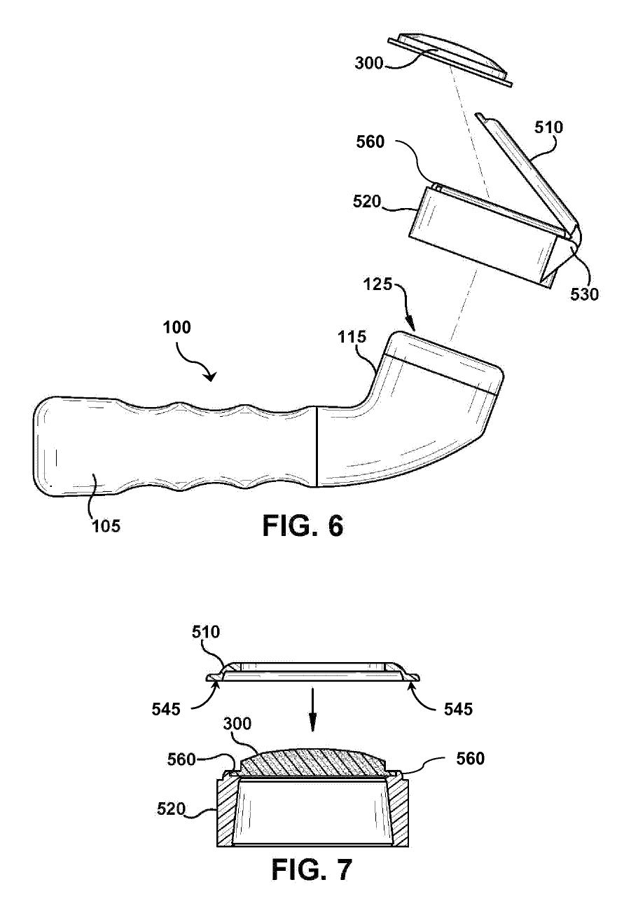

The base 520 is hollow so that when attached to the head of an ultrasound device, the transducer surface (applicator surface that is applied to a patient) of the ultrasound device is positioned against the bottom surface of the solidified gel 300. See for example, applicator surface 125 in FIG. 1A or FIG. 6, where the nose 120 (or head 115) and surface 125 would be inserted into the mounting cavity 550. Thus the solidified gel 300 (its bottom surface) is pressed into contact against the applicator surface 125 of the ultrasound device to reduce or eliminate air in between.

FIG. 5F illustrates a side view of the attachable adaptor 500 of FIGS. 5D and 5E with the cap 510 closed and locked against the gel receiving end of the base 520. Thus, the solidified gel 300 is locked in between the cap 510 and the base 520. The gel 300 cannot fall out unless the cap 510 is opened. Since the solidified gel 300 is formed with a thickness greater than the thickness of the cap 510, a portion of the solidified gel 300 extends through the opening 515 of the cap 510. Thus the solidified gel 300 is exposed for contacting an ultrasound target and functions as a conductive medium for ultrasound energy transmitted by the ultrasound device.

FIG. 5G illustrates a side view in cross-section of the attachable adaptor 500 of FIG. 5E without the solidified gel 300 positioned therein. Since the gel 300 is not shown, FIG. 5G shows the gel receiving end without being obstructed by the gel 300. For example, the gel receiving end of the base 520 includes a side wall 560 formed from the housing and defines the gel cavity (labeled as gel cavity 570). The gel cavity 570 is formed to receive a piece of solidified gel (e.g., gel 300). The side wall 560 extends around the perimeter of the housing as a continuous wall or may include multiple smaller side walls that are formed along the perimeter in intervals.

In one embodiment, the gel receiving end of the base 520 includes an inner ledge 580. The inner ledge 580 is formed around the perimeter of the housing (e.g., along and extending from the side wall 560) so that the solidified gel 300 is held by the ledge 580 at least along the locking ring 305 portion of the gel 300. This is seen, for example, in FIGS. 5A and 5E where the locking ring 305 sits on the ledge 580. When the cap 510 is closed and locked to the base 520, the cap 510 engages the gel locking ring 305 (e.g., edges of the gel 300) and presses the gel locking ring 305 against the ledge 580 to lock the gel 300 in place. The outer edges of the solidified gel (locking ring 305) are between the inner ledge 580 and a portion of the cap frame 510.

In another embodiment, the gel receiving end of the base does not include the inner ledge 580. Instead the side wall 560 extends around the perimeter of the base 520 to form the gel receiving cavity 570. When the adaptor 500 is attached to an ultrasound device, the bottom surface of the solidified gel 300 sits on the applicator surface of the ultrasound device (see for example, FIG. 6 and applicator surface 125). This embodiment is also similar to that shown in FIG. 8 where the side wall 560 defines the gel cavity 570 and a solidified gel (with elongated shape to fit into the gel cavity 570) would be positioned against the applicator surface 125 within the gel cavity 570. The applicator surface 125 serves as a floor for the gel 300 and the side wall 560 prevents side-to-side movement of the gel 300. Thus when the cap 510 is locked onto the gel receiving end, the cap 510 presses (clamps) the gel locking ring 305 and prevents the gel 300 from falling out of the gel cavity 570.

FIG. 6 illustrates one embodiment of an assembly view of the adaptor 500 from FIG. 5A being attached to the head/nose 115 of an ultrasound device/probe 100 (e.g., the device 100 from FIG. 1A). Once attached, the adaptor 500 transforms the flat applicator surface 125 (and head 115) to a surface that includes (i) a gel cavity and (ii) an opening and closing mechanism (cap 510) for locking solidified gel against the applicator surface 125. As described previously, the cap 510 is configured to close and lock the solidified gel 310 to the head 115 of the ultrasound device 100. Thus, when the ultrasound device 100 is applied against a target (e.g., skin) and is slid around the target, the solidified gel 300 is held in place. The cap 510 is also configured to open and unlock allowing the solidified gel 300 to be removed and replaced with another piece of solidified gel. As previously described, in one embodiment, a locking and unlocking mechanism includes latch tabs (on the cap 510 or on the base 520) and corresponding latch recesses (on base 520 or cap 510) that snap fit together, press fit together, hook together, or otherwise mate together and connect.

FIG. 7 illustrates a side view of another embodiment of the attachable adaptor of FIGS. 5A-5G with a removable cap/top portion 510 for locking the solidified gel 300 to the base portion 520. Here, the cap 510 is not secured to the base 520 with a hinge but is a separate component and is removable. The cap 510 and base 520 are a two piece mechanism for locking gel. Thus the cap 510 is a separable component from the base 520. The cap 510 and the base 520 and/or the side wall 560 include the locking elements of corresponding latch tabs and latch recesses 540 as previously described.

To connect the top portion (cap) 510, the top portion 510 is snapped/latched on the top surface of the base 520 with one or more snap/latch tabs 545 as previously described. The latch tabs 545 are configured to mate with and locking with corresponding latch recesses 540 (see FIG. 5B) formed on the side wall 560 of the base 520 or housing of the base 520. Thus locking in the solidified gel 300. The cap 510 is disconnected and removed from the base 520 by disengaging the latch tabs 545 to open and release the gel 300. Of course, the latch tabs 545 and latch recesses 540 can be reversed.

In another embodiment, the cap 510 is connected to the base 520 via one or more columns or posts. Rather than pivoting along a hinge 530 as in FIG. 5A, the cap 510 moves vertically up and down with the posts relative to the gel receiving surface of the base 520 to open (disconnected from base 520) or close the cap 510 (connect and lock to the base 520). In one embodiment, the posts are configured to retract into recesses formed in the housing side walls of the adaptor base 520. The posts and recesses may be spring loaded in one embodiment.

FIG. 8 illustrates another embodiment of the ultrasound device/probe 100 from FIG. 1A with a differently shaped body/housing, handle 105, and with an elongated head 115 extending from the handle 105, and elongated applicator surface 125 (as compared to the circular surface 125 in FIG. 1A). In FIG. 8, the ultrasound device 100 includes the gel cavity 570 and the cap 510 of the adaptor 500 formed as part of the head 115 (integral together). The housing of the head 115 also forms the side wall 560 that defines the gel cavity 570. For example, the side wall 560 is molded as part of the housing of the head 115.

The embodiments of FIGS. 5-7 show the adaptor 500 as a component that retrofits onto an ultrasound device/probe that does not have a gel cavity as part of the head 115. The ultrasound device 100 of FIG. 8 is not a retrofit. However, once the adaptor 500 of FIGS. 5-7 is attached to an ultrasound device, the resulting device has the same or similar features as the device 100 in FIG. 8 that is integrally formed with the gel locking mechanism of the adaptor 500.

With reference to FIG. 8, the head 115 is formed with the side wall 560 that extends out from the end of the head 115 and around the perimeter of the head (around the perimeter of the applicator surface 125). The side wall 560 forms and defines the gel cavity 570 around the applicator surface 125. An elongated piece of solidified gel having bottom surface that corresponds to and fits into the gel cavity 570 would be used. The solidified gel, once inserted into the gel cavity 570, sits against the applicator surface 125.

The gel locking mechanism (cap 510) is connected to the side wall 560 and/or head 115 with one or more hinges 530. With the hinge 530, the cap 510 is a flip-top. In another embodiment, the cap 510 is connected to the head 115 as a flip-top by being molded together at a portion of the cap 510 with a flexible element (e.g., a flap, a cord, flexible portion of the housing connected to the cap 510 that allows the cap 510 to open and close). Thus in some embodiments, the housing includes means for connecting the cap 510 with the housing (e.g., head 115) that includes one or more hinges, retractable posts, a flap, a cord, a flip-top, or an equivalent mechanism that allows the cap 510 to be connected to the head 115 and move relative to the head 115.

In one embodiment, the cap 510 is formed with a frame that corresponds in shape to the shape of the gel cavity 570. As such, the frame of the cap 510, when closed, encloses the sides of the gel cavity 570 and is in contact with the outer edges of the solidified gel (while inserted in the gel cavity 570). As previously described, the cap 510 includes latch tabs for connecting to corresponding latch recesses in the side wall 560 or vice versa.

The cap 510 is configured to pivot at the hinge 530 and can close onto the head 115 to lock in a piece of solidified gel (that is within the gel cavity 570) against the applicator surface 125. The top portion (central portion) of the gel would extend through and be exposed through the opening 515 of the cap 510 as previously described, while portions of the cap frame 510 are in contact with (clamp down on) the outer edges of the solidified gel. In another embodiment, the cap 510 is not connected to the side wall 560 by the hinge 530 but the cap 510 is a separate separable component as a two piece mechanism (as previously described with reference to FIG. 7) and locks with and unlocks from the side wall 560 as previously described (e.g., with latch tabs and latch recesses).

Although not shown, the head 115 includes at least one transducer mounted within the housing and in close proximity to the inner side of the applicator surface 125. The ultrasound device 100 includes ultrasound device components as previously described or other ultrasound components for generating ultrasound energy as known in the art.

### DEFINITIONS

The following includes definitions of selected terms employed herein. The definitions include various examples and/or forms of components that fall within the scope of a term and that may be used for implementation. The examples are not intended to be limiting. Both singular and plural forms of terms may be within the definitions.

References to “one embodiment”, “an embodiment”, “one example”, “an example”, and so on, indicate that the embodiment(s) or example(s) so described may include a particular feature, structure, characteristic, property, element, or limitation, but that not every embodiment or example necessarily includes that particular feature, structure, characteristic, property, element or limitation. Furthermore, repeated use of the phrase “in one embodiment” does not necessarily refer to the same embodiment, though it may.

The term “conductive medium” is used to refer to a substance that is used during an ultrasound procedure that assists in coupling the ultrasound device/probe head or applicator tip to a subject/target (e.g., the skin of a patient or other surface) and conducts ultrasound energy. Typically, the conductive medium is ultrasound gel but other substances can be used such as shampoo, hairstyling gel, hand lotion, hand sanitizer, liquid dishwashing detergent, olive oil (or other oil based substances), or other substance with a composition that is appropriate to function with an ultrasound device. Many substances can form gels when a suitable thickener or gelling agent is added to their formula to change the viscosity. These substances, which ever one is chosen, are preconfigured/formed into a solidified state as an individual piece of conductive medium as explained previously (e.g., solidified state may be any state in which the gel maintains its shape and dimensions, and does not flow). References to the term “gel” is intended to refer to any of these conductive media in a solidified form that is appropriate for an ultrasound procedure.

While example devices and methods have been illustrated by describing examples, and while the examples have been described in considerable detail, it is not the intention of the applicants to restrict or in any way limit the scope of the appended claims to such detail. It is, of course, not possible to describe every conceivable combination of components or methodologies for purposes of describing the systems, methods, and so on described herein. Therefore, the disclosure is not limited to the specific details, the representative apparatus, and illustrative examples shown and described. Thus, this application is intended to embrace alterations, modifications, and variations that fall within the scope of the appended claims.

To the extent that the term “includes” or “including” is employed in the detailed description or the claims, it is intended to be inclusive in a manner similar to the term “comprising” as that term is interpreted when employed as a transitional word in a claim.

To the extent that the term “or” is used in the detailed description or claims (e.g., A or B) it is intended to mean “A or B or both”. When the applicants intend to indicate “only A or B but not both” then the phrase “only A or B but not both” will be used. Thus, use of the term “or” herein is the inclusive, and not the exclusive use.

What is claimed is:

1. An adaptor device for attachment to an ultrasound device, the adaptor device comprising:
a housing formed with a mounting end and a gel receiving end;
a mounting cavity formed in the mounting end, wherein the mounting end is shaped to mount onto an end of the ultrasound device;
wherein the gel receiving end includes a side wall formed from the housing that defines a gel cavity for receiving a solidified gel;
a cap formed with a frame and having a central opening defined therethrough, wherein the frame is configured to lock to and unlock from the gel receiving end of the housing; and
wherein in a locked position, the cap is closed onto the gel receiving end and the central opening allows the solidified gel inserted into the gel cavity to have a portion extend through the central opening.

2. The adaptor device of claim 1, wherein the housing and the cap are connected by at least one hinge, wherein the cap is configured pivot at the at least one hinge.

3. The adaptor device of claim 1, wherein the housing includes a means for connecting the cap to the housing.

4. The adaptor device of claim 1, wherein the gel receiving end includes an inner ledge formed within the housing, wherein the inner ledge is configured to hold the solidified gel when the solidified gel is inserted into the gel cavity;
wherein a portion of the frame of the cap overlaps outer edges of the solidified gel when the solidified gel is inserted into the gel cavity and the cap is in the locked position, and
wherein the outer edges of the solidified gel are between the inner ledge and the portion of the frame.

5. The adaptor device of claim 1, wherein the cap includes one or more latch tabs formed on the frame and wherein the gel receiving end of the housing includes one or more latch recesses positioned to connect with the one or more latch tabs;
wherein the frame is configured to lock to and unlock from the gel receiving end of the housing by connecting the one or more latch tabs to the one or more latch recesses.

6. An adaptor with a locking mechanism for an ultrasound device, the adaptor comprising:
a housing configured to attach the adaptor to a head of the ultrasound device;
a cavity formed by the housing that is configured to receive and contain an ultrasound conductive medium that is in a solidified form; and
a hinged locking mechanism for locking the solidified form of the ultrasound conductive medium into the cavity by closing the hinged locking mechanism over the cavity and against the housing.

7. The adaptor of claim 6, wherein the hinged locking mechanism is configured to unlock the solidified form of the ultrasound conductive medium from the cavity by disconnecting the hinged locking mechanism from the housing.

8. The adaptor of claim 6, wherein the hinged locking mechanism includes a cap formed with a frame and having a central opening formed through the frame, wherein the frame is connected to the housing by at least one hinge.

9. The adaptor of claim 6, wherein the hinged locking mechanism includes one or more latch tabs and wherein the housing of the adaptor includes one or more latch recesses corresponding to the one or more latch tabs;
wherein the hinged locking mechanism is configured to lock to and unlock from a side wall of the housing by connecting the one or more latch tabs to the one or more latch recesses.

10. The adaptor of claim 6, wherein the cavity includes an inner ledge formed by the housing, wherein the inner ledge extends around a perimeter of the cavity and is configured to hold the solidified form of the ultrasound conductive medium when the solidified form is inserted into the cavity.

11. The adaptor of claim 6, wherein the adaptor is a retrofit component for attachment to the ultrasound device.

12. An ultrasound device with a gel locking mechanism, the ultrasound device comprising:
a housing including a handle and a head extending from the handle;
an applicator surface formed on the head;
at least one transducer for generating ultrasound energy, wherein the at least one transducer is mounted within the housing and adjacent the applicator surface; and
a gel locking mechanism comprising:
a side wall extending from the head and around a perimeter of the applicator surface where the side wall defines a gel cavity; and
a cap formed with a frame and having a central opening defined therethrough, wherein the frame is configured to lock to and unlock from the side wall; and
wherein in a locked position, the cap is closed onto the side wall to secure a piece of solidified gel that is inserted into the gel cavity and wherein the central opening allows the solidified gel inserted into the gel cavity to have a portion extend through the central opening.

13. The ultrasound device of claim 12, wherein the head and the cap are connected by at least one hinge.

14. The ultrasound device of claim 12, further including the solidified gel mounted within the gel cavity; and
wherein in the locked position, the frame of the cap is connected to the side wall and encloses at least sides of the gel cavity and is in contact with outer edges of the solidified gel.

15. The ultrasound device of claim 12, wherein the cap includes one or more latch tabs formed on the frame and wherein the side wall of the housing includes one or more latch recesses corresponding to the one or more latch tabs; and
wherein the frame is configured to lock to and unlock from side wall of the housing by connecting the one or more latch tabs to the one or more latch recesses.

16. The ultrasound device of claim 12, wherein the cap is connected to the housing as a flip-top.

17. The ultrasound device of claim 12, wherein the gel locking mechanism is an adaptor device that is attachable to the head of the ultrasound device and detachable from the head.

18. The ultrasound device of claim 12, wherein the side wall is formed as part of the housing and extends from the head; and
wherein the cap is connected for movement to the housing of the ultrasound device or is a separable component from the housing.

19. The ultrasound device of claim 12, wherein the gel locking mechanism includes a means for connecting the cap to the housing of the ultrasound device.
